(19) United States
(12) Patent Application Publication
(10) Pub. No.: US 2008/0261909 A1
(43) Pub. Date: Oct. 23, 2008

(54) Gene Delivery to Organs

(75) Inventors: J. Kevin Donahue, Rocky River, OH (US); Amy D. McDonald, Sykesville, MD (US); Kan Kikuchi, (US)

Correspondence Address: BANNER & WITCOFF, LTD., 1100 13th STREET, N.W., SUITE 1200, WASHINGTON, DC, 20005-4051, US

(73) Assignee: The John Hopkins University, Baltimore, MD (US)

(21) Appl. No.: 12/029,020

(22) Filed: Feb. 11, 2008

Related U.S. Application Data
(63) Continuation of application No. 10/859,604, filed on Jun. 3, 2004, now abandoned.
(60) Provisional application No. 60/475,843, filed on Jun. 4, 2003.
(60) Provisional application No. 60/488,567, filed on Jul. 17, 2003.

(51) Int. Cl. A61K 31/7088; A61P 43/00

(52) U.S. Cl. 514/44

(57) ABSTRACT

Application of a virus with poloxamer alone onto atria results in diffuse epicardial gene transfer with negligible penetration into the myocardium. Progressive increases in protease concentration, however, allow transmural gene transfer. After protease exposure, echocardiographic left atrial diameter does not change. Left atrial ejection fraction decreases on post-operative day 3, but returns to baseline by day 7. At appropriate protease concentrations, tissue tensile strength is unaffected by the procedure. Transmural atrial gene transfer can be effected using this direct “painting” method.

This application is a continuation application of U.S. application Ser. No. 10/859,604 filed Jun. 3, 2004 which claims priority to provisional Application Ser. No. 60/475,843 filed Jun. 4, 2003 and provisional Application Ser. No. 60/488,567, filed Jul. 17, 2003.

This invention was made using funds from the United States government (NIH grant HL67148). Therefore the United States government retains certain rights in the invention.

### FIELD OF THE INVENTION

The invention relates to the field of nucleic acid delivery, both in vivo and ex vivo. In particular, it relates to delivery of nucleic acids to whole organs.

### BACKGROUND OF THE INVENTION

Advances in molecular diagnosis and therapy have raised the possibility of curing common diseases. The molecular identities of several key proteins have been discovered, and the corresponding genes have been transfected in vitro to further evaluate function.¹ In a limited way, these genes have been introduced in vivo to observe changes in function.²⁻⁴ The major impediment to widespread use of molecular therapies has been the absence of effective and safe delivery methods for homogeneous, high-density whole organ gene transfer. This deficit has limited existing gene therapy clinical trials to focal applications (e.g., secreted particles such as angiogenic factors, regulators of cell cycling, or clotting proteins)⁵⁻⁷

A major frustration in the development of gene transfer strategies has been the lack of effective, clinically relevant delivery methods. Currently available options include direct injection of vectors, perfusion of vectors through coronary arteries or veins, and pericardial administration.^{3,16,17} The major weakness of direct injection is the limited distance that the vector travels from the delivery site. Typically gene transfer occurs only within a few millimeters of the injection,¹⁸ which is unacceptable for whole atrial or whole ventricular applications. Virus perfusion of the coronary vasculature results in diffuse gene transfer to as many as 75% of cardiac myocytes, but the effect cannot be isolated to atria or ventricles, and the harsh conditions necessary to achieve this level of gene transfer include hypothermia to 18° C., perfusion with large doses of vascular permeability agents, and disruption of the normal coronary blood flow for several minutes.¹⁹ Such conditions are not easily transferable to the clinic. Pericardial delivery has until now been limited by the inability to penetrate through the epicardial layer in a safe and controlled fashion.^{17,20}

There is a need in the art for a gene transfer method that results in homogenous, high density gene uptake by whole organs.

### BRIEF SUMMARY OF THE INVENTION

In a first embodiment of the invention a method is provided for delivering a nucleic acid to an organ. A composition is applied to the external surface of an organ. The composition comprises a nucleic acid, a poloxamer, and a protease.

In a second embodiment of the invention a composition is provided. The composition comprises a nucleic acid, a poloxamer, and a protease.

In a third embodiment of the invention a kit is provided. The kit comprises a poloxamer and a protease, wherein the poloxamer and the protease are in a divided or undivided container.

### BRIEF DESCRIPTION OF THE DRAWINGS

FIGS. 1A-D show stained specimens demonstrating β-galactosidase gene transfer to the porcine cardiac atria. (FIG. 1A) Gross and microscopic sections from left atrium excised 3 days after painting with Ad gal, 0.5% trypsin and 20% poloxamer. Myocytes expressing β-gal show characteristic blue stain. Magnification ×400. (FIG. 1B) Gross and microscopic sections of a left atrium excised 3 days after painting with AdHERG-G628S, 0.5% trypsin and 20% poloxamer for control. (FIG. 1C) Percentage of β-gal positive cells in epicardial, mid-myocardial and endocardial layers based on trypsin concentration. (n=5 in each group) (FIG. 1D) Hematoxilin-eosin stain after 3 days painting with Adβgal, 0.5% trypsin and 20% poloxamer shows pericardial adhesions and epicardial inflammation on the right, but no penetration of the inflammatory infiltrate into the myocardium. Magnification ×100.

FIGS. 2A-2B show echocardiographic data of (FIG. 2 A) left atrial diameter and (FIG. 2 B) LAEF were measured 3 days after painting with each trypsin concentration (left panel, ANOVA p<0.01). Both parameters were measured serially over 21 days after painting with the 0.5% trypsin concentration (right panel). (n=5 for 3 day studies and n=3 for 21 day studies, *p<0.01).

FIGS. 3A-3B shows effects on tensile strength. (FIG. 3 A) The effect of trypsin on the tensile strength of atrial myocardium 3 days after painting was assessed by tensile strength test. (n=5 in each group, *p<0.05) (FIG. 3 B) Longitudinal effects of the painting method on tensile strength. Comparison is made between normal controls (n=5), 3 day post-0.5% trypsin exposure (n=5), and 21 day post-0.5% trypsin exposure (n=6). No significant differences are noted between groups.

FIGS. 4A-4F. (FIG. 4A) Diagram showing 10 atrial areas used for monophasic action potential duration (MAP) measurements. (FIG. 4B) Atrial MAPD90 measurements on days 0 and 21 in AdHERG-G628S infected hearts (n=3, *p<0.05). (FIG. 4C) Ventricular MAPD90 measurements from the basal region of the ventricles adjacent to atrial areas 4 and 9. (FIG. 4D) Examples of atrial and ventricular MAP recordings. (FIG. 4E, FIG. 4F) Atrial and ventricular effective refractory period (ERP) measurements before and 21 days after AdHERG-G628S.

### DETAILED DESCRIPTION OF THE INVENTION

It is a discovery of the present inventors that homogeneous, high-density, whole organ gene transfer can be achieved using a combination of a poloxamer and a protease to deliver a nucleic acid. The components of the combination may be applied serially or simultaneously to the whole organ, i.e., the components can be pre-mixed or mixed on the organ itself.

Nucleic acids which can be used in the present invention are any which provide a desired function. The nucleic acids may be protein-encoding, or they may be anti-sense or triple-strand forming. The nucleic acids may encode small inhibitory RNA molecules or anti-sense RNA molecules. The function of the protein can be to improve a function of the organ or organism being treated. The encoded protein can add a function not possessed by the organ. The function of an encoded protein can be to label or mark particular types of cells. The nucleic acids can be packaged in a virus or other protein or polymer package. The nucleic acids can be in liposomes or other small particles. Naked nucleic acids can also be used. Any type of virus can be used which infects the cells of the target organ. Such viruses include, but are not limited to adenoviruses, helper-dependent adenoviruses, adeno-associated viruses, retroviruses, and herpes simplex viruses. The genes can be placed under the control of regulatable promoters. These can be used to obtain temporally or spatially desired regulation. Thus a promoter can be used which is specific for a particular organ or cell type. Alternatively, a promoter can be used which can be controlled by administration of an exogenous agent or a temporally regulated endogenous agent.

Poloxamers (or PLURONICS™) are synthetic polymers with a variety of unique properties: they are formed from alternating blocks of ethylene oxide and propylene oxide polymers. They are nontoxic and inert surfactants. They have been used as food additives and in pharmaceutical preparations. They form micelles with hydrophobic cores and hydrophilic exteriors. They are liquid at colder temperatures, polymerizing to form a gelatinous matrix at body temperature (for review see Kabanov et al.²¹). Typical poloxamers include L61, F127, P108, P188, P238, P338, and P407. Any poloxamer can be used in the invention. Suitable concentrations of poloxamer may range from 5 to 30%, from 15 to 25%, or from 18 to 23%.

Any proteases can be used in the present invention, so long as they do not cause unacceptable toxicity to the recipient tissues, and do not unacceptably impair the gene transfer rate due to destruction of the delivery vehicle or the cell surface receptor used by the vector for attachment or entry into target cells. Suitable proteases which can be used include trypsin and chymotrypsin. Other suitable proteases include matrix metalloproteases, hyaluronidase, and collagnase. Suitability of a protease can be determined by routine testing of the delivery vehicle and the receptor for protease resistance. Subtilisin and pronase cleave the adenovirus receptor from the cell and therefore should be avoided when using adenovirus as a delivery vehicle. The inventors have found that the concentration of protease is critical for successful and safe nucleic acid delivery. For trypsin, it has been found that ranges between 0.05 and 1% or between 0.1 and 0.5% provide excellent nucleic acid delivery and do not damage a recipient organ. Other proteases can be similarly tested to determine the optimal concentrations for use.

The gene transfer method of the present invention results in homogenous, high density gene uptake. This method can be used for hearts as well as in other thin-walled organs or structures, such as hollow organs, including gastrointestinal organs or reproductive organs. The organ may be, for example, the stomach, gall bladder, small intestine, large intestine, rectum, uterus, or urinary bladder. The organ may be any thin-walled organ, including but not limited to the eye, skin, diaphragm, and lung. The method can be used, inter alia, for humans undergoing cardiac surgical procedures. Thoracoscopic delivery can optionally be used to substantially reduce the invasive nature and increase subject tolerance.

Several investigators have demonstrated the ability of poloxamer solutions to hold DNA or recombinant viruses in localized areas, thus increasing the time available for gene transfer. This innovation was originally reported by March et al.⁸ In cultures of vascular smooth muscle cells, they showed a 10-fold increase in gene transfer after complexing the virus with poloxamer P407 (which has the same structure as the F127 used in this study). Feldman et al. extended these observations to an in vivo model of rat carotid artery delivery.⁹ After balloon injury of the carotid, infusion of adenoviruses complexed with P407 allowed a 50% reduction in vector delivery time with no reduction in gene transfer efficacy. Lemieux et al. used poloxamers L61 and P407 to achieve the same effect, suggesting that the improved gene transfer efficiency is a class effect.²²

Lonberg-Holm and Philipson reported that cultures of HeLa cells could be more efficiently infected with adenovirus after exposure to trypsin or chymotrypsin.¹⁰ The effect was not universal, however, because exposure to pronase or subtilisin eliminated adenovirus attachment. The conclusion from these studies was that pronase and subtilisin cleaved the receptor and prevented virus attachment, while trypsin and chymotrypsin cleaved other surface proteins improving access to the receptor. Fromes et al. extended these findings by including collagenase and hyaluronidase in gene transfer solutions injected into the pericardial space of rats.²³ They observed patchy gene transfer to the ventricular epicardium and to some myocytes penetrating to a depth of roughly half-thickness of the ventricular myocardium. As such, the gene transfer was not transmural; it did not affect 100% of myocytes in any layer, and it was not specific for any portion of the rat heart. There was no comment on any atrial gene transfer with this delivery technique.

Our data demonstrate that painting recombinant adenoviruses onto a target increases the specificity of delivery, that complexing the virus with poloxamers improves the gene transfer efficiency, and that including trypsin in the solution allows transmural penetration. No loss of infectivity was observed in the trypsin solutions, suggesting that the adenoviral particle is not susceptible to degradation by trypsin. Safety concerns are allayed by documentation of preserved atrial size and tensile strength for a trypsin concentration of 0.5%, a concentration sufficient to achieve transmural gene transfer. A transient decrease in LAEF was observed, but this measure recovered by day 7. Unlike all other delivery methods, no evidence of reporter gene expression was found in the cardiac ventricles, lungs, liver, spleen, kidneys, gonads or skeletal muscle. Epicardial adhesions and inflammation are concerning to the same extent that they would be for any open-chest surgery. No increase was noted in connection with the painting procedure. Importantly, despite the invasive nature of the delivery method, no adverse events were noted in the study population. Furthermore, we demonstrated that functional properties of the atrial muscle could be modified using this gene transfer method. HERG-G628S gene transfer caused significant prolongation of atrial action potential duration and effective refractory period.

Long-term expression vectors (helper-dependent adenoviruses²⁴ or adeno-associated viruses²⁵) and/or less invasive delivery methods (e.g., thoracoscopy) can be used to treat common diseases such as atrial fibrillation or sinus node dysfunction.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### EXAMPLES

### Example 1

### Materials and Methods

Adenoviruses and solutions: Recombinant E1, E3-deleted adenovirus expressing the reporter gene E. coli β-galactosidase (Adβgal) was a gift from Dr. Frank Graham; the vector contained the E. coli lac Z gene driven by the human cytomegalovirus immediate early promoter. A plasmid containing HERG-G628S was a gift from Dr. Eduardo Marbán. AdHERG-G628S was constructed using the Cre-lox system as previously reported.¹¹ The resulting virus was plaque purified, expanded and characterized as previously described,² and stored in phosphate-buffered saline (PBS) with 10% glycerol at −70° C. Virus titers were determined by the average of two plaque assays. Virus stocks were free of replication-competent adenovirus when tested with a supernatant rescue assay that has the sensitivity to detect one replication-competent virus in 10⁹ recombinant viruses.

Infection solutions were made by adding trypsin at the appropriate concentration to PBS. The resulting solution was chilled to 4° C. and added slowly to polyoxyethylene/polyoxypropylene block co-polymer F127 (also known as poloxamer or pluronic, F127 or P407, BASF Corporation, Mount Olive, N.J.). The total volume was 5 ml. After the poloxamer had dissolved into solution, the mixture was warmed at 37° C. to achieve a gel-like consistency, because the gel provided more consistent coverage for painting. Immediately before use, the adenovirus stock solution was added to the poloxamer/trypsin/PBS solution for a final virus concentration of 1×10⁹ pfu/ml.

Gene Transfer Procedure: Thirty-five domestic pigs underwent a 3 days protocol that evaluated the effect of trypsin concentration and virus delivery. Five pigs each were included in the following experimental categories: sham-operated controls, Adβgal solution without trypsin/poloxamer, Adβgal solution with 20% poloxamer and 0, 0.1, 0.5, or 1% trypsin, and AdHERG-G628S with 20% poloxamer and 0.5% trypsin. An additional 6 pigs were observed for 21 days after delivery of adenovirus (3 Adβgal and 3 AdHERG-G628S) in 20% poloxamer and 0.5% trypsin.

Domestic pigs (20-30 kg) were sedated with ketamine (30 mg/kg) and anesthetized with sodium thiopental (2-5 ml of 5% solution). Anesthesia was maintained with 0.5-2% isoflurane. After sterile preparation, the chest was opened via conventional median sternotomy. The pericardium was incised to expose both atria. With this method, the pleurae remained intact, and the lung fields were never exposed. A total of 5 ml of adenoviral solution was painted onto both atria using a rounded bristle, flat paintbrush composed of camel hair. The heart was manipulated to expose all epicardial surfaces of the atria. Each atria was coated twice for 30 seconds each, and approximately 60 seconds was given between coats of painting to allow adsorption.

After painting, the atria were left exposed to air for a 2 minute period to allow drying of the solution. Animal body temperature was maintained by use of a heat pump (Gaymar, N.Y., USA). At the conclusion of the procedure, the sternum was closed with stainless steel wires; air was evacuated from the mediastinum, and the subcutaneous tissue and skin were sutured. The animals received typical post-operative care, including narcotics for pain management. All animals survived the procedure, and their post-operative course was unremarkable.

The animals used in this study were maintained in accordance with the guiding principles of the American Physiological Society regarding experimental animals. The experimental protocol was approved by the Institutional Animal Care and Use Committee at the Johns Hopkins University.

Tissue processing: After 3 or 21 days, animals were sedated with ketamine (30 mg/kg) and anesthetized with sodium thiopental (2-5 ml of 5% solution). Anesthesia was maintained with 0.5-2% isoflurane. The chest was re-opened, the animals were given heparin (10,000 units), and then euthanized with overdose of KCl. The heart and lungs, and sections of liver, kidney, spleen, skeletal muscle and gonad were removed and rinsed with PBS. This protocol was followed to allow quick removal of all organs to minimize protein degradation. Sections of each atria were excised for tensile strength testing as described below. Multiple sections of all organs were frozen, cut to 5 μm thickness using a microtome, fixed with a solution of 2% formalin and 0.2% glutaraldehyde and stained with X-gal or with Hematoxilin-Eosin (HE) using conventional methods. The remaining portions of atria, ventricles and non-cardiac organs were fixed in a solution of 2% formalin, 0.2% glutaraldehyde for 15 minutes at room temperature, rinsed 3 times with PBS, and stained with X-gal solution so that both microscopic and gross tissue staining could be observed in the same animals. X-gal staining was performed at pH 8 to minimize non-specific staining.¹²

Quantification of infection efficiency: The tissue was divided into thirds to define the epicardial, mid-myocardial and endocardial layers. The percentage of cells expressing β-galactosidase was determined by counting cells in 5 randomly selected fields throughout each left and right atrium for each tissue layer (100 cells per field, 500 cells per atrial layer).

Measurement of tissue structure and function: Echocardiographic examinations were performed one day prior to and 3 days after gene transfer. For the 21 day experiments, further echocardiograms were performed at 7, 14, and 21 days after gene transfer. Ejection fraction of the left atrium was calculated from a right parasternal approach. Chamber volume was automatically calculated from the short axis of the left atrium during systole and diastole using software provided with the system (Agilent 5500, MA, USA).

Measurement of tissue tensile strength: Structural integrity of the tissue was evaluated by the progressive addition of force to the tissue to define the breaking point. Strips of atria were cut to a uniform size (10 mm long, 2 mm wide, and 3.5 mm thick wall) and attached to the tensile strength apparatus. Force on the non-fixed end of the tissue was progressively increased at a fixed rate (0.015 N/sec for 5 sec intervals followed by 10 sec pauses) to define the breaking point of the strip of tissue.

Monophasic action potential recording and electrophysiological measurements: For the 21 day experiments, during the open-chest gene transfer and tissue harvest procedures, monophasic action potential (MAP) and effective refractory period (ERP) measurements were assessed from the epicardial wall under visual control to reproduce the location. MAPs were acquired in digital format using a Cardiac Pathways Corporation system (Sunnyvale, Calif., USA). The atrial and ventricular ERP were measured by programmed stimulation with a drive train cycle length of 400 ms. MAPs were recorded using a 7-French MAP/Pacing catheter (Boston Scientific, Natick, Mass.), which was positioned at the epicardial free wall of both atrium and ventricle. The MAP duration was measured as the interval from the steepest part of the MAP upstroke to the level of 90% repolarization (MAPD90).¹³ MAPs were recorded during regular pacing with a drive train cycle length of 400 ms.

Statistical analysis: The data are presented as mean ±s.e.m. Statistical differences were determined using Student's t-test and repeated measures ANOVA, where appropriate. A p value of <0.05 was considered statistically significant.

### Example 2

### Gene Transfer Efficiency

To test the hypothesis that polyoxyethylene/polyoxypropylene block co-polymers (poloxamers) would improve gene transfer efficiency by prolonging atrial-virus contact time and that trypsin would increase virus penetration, we developed a gene transfer vector application method that involved painting solutions containing 20% poloxamer F127, 1×10⁹ pfu/ml Adβgal and varying concentrations of trypsin onto the epicardial surface of pig atria. Control groups included animals receiving open-chest manipulation of the heart without painting and those receiving the painting procedure with AdHERG-G628S, a gene that does not induce β-galactosidase production. Gene transfer efficacy and safety were tested at 3 and 21 days. The 3 day time point was chosen to observe acute effects of the painting process, and the 21 day point was chosen to see longer lasting effects from the method. The 20% poloxamer concentration was chosen after trials with concentrations from 5-30% showed that 20% most effectively went into solution at 4° C. and formed a gel at body temperature. In this proof of concept work, observations were not extended beyond 21 days to avoid confounding by the loss of transgene expression that has been well documented with first generation adenovirus vectors (for review see Kovesdi et al.¹⁴).

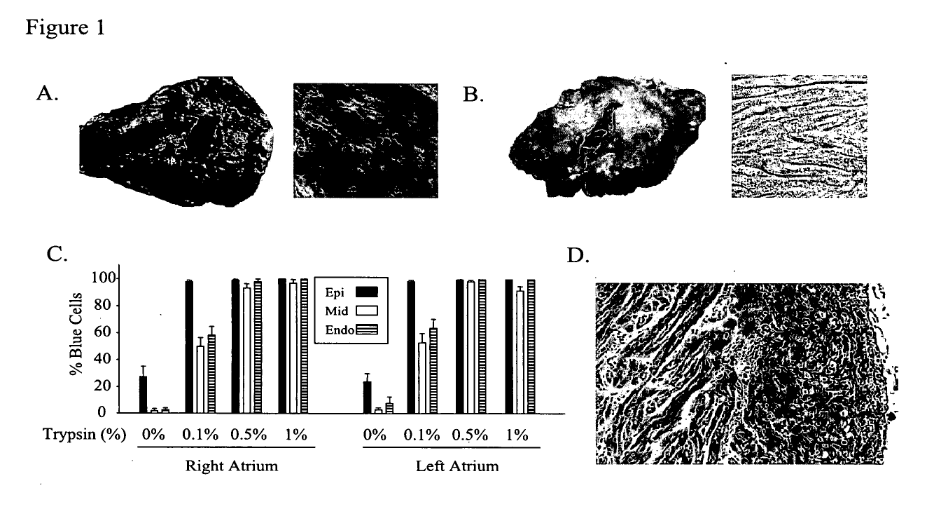

Initial studies applying 5 ml of virus solution containing 1×10⁹ pfu/ml of Adβgal in the absence of poloxamer or trypsin showed scattered areas of epicardial gene transfer and no penetration into the tissue (data not shown). Addition of 20% poloxamer to the virus solution allowed homogeneous epicardial gene transfer but penetration of the virus into the myocardium was still negligible. Trypsin at a concentration of 0.1% allowed marginally better penetration, and concentrations of 0.5% and 1% allowed complete transmural gene transfer (FIG. 1A). Control animals undergoing the same procedures without addition of virus to the solution or with addition of AdHERG-G628S failed to show any blue staining (FIG. 1B).

Microscopic evaluation of tissue sections confirmed the observations from the gross examination (FIG. 1). The blue coloration was contained within the myocytes for the Adβgal animals, further verifying that gene transfer and not inflammation was responsible for the color change. Neither sham nor AdHERG-G628S control groups had any microscopic blue staining, indicating that the blue coloration in the active treatment group was specific for effective gene transfer. The 0% trypsin group had negligible gene transfer beyond the epicardium. The 0.1% trypsin sections had detectible β-gal activity in approximately half of the mid-myocardial and endocardial cells, and both 0.5% and 1% trypsin treatments had complete transmural gene transfer (FIG. 1C). Microscopic evaluation of the atria from the 21 day poloxamer/0.5% trypsin experiments demonstrated persistence of 100% transmural gene expression at that time point.

### Example 3

### Gene Transfer Safety

Safety of the painting method was assessed by histological analyses, serial echocardiography, and tensile strength testing. Histological analyses included X-gal staining for non-target gene transfer and H&E staining for inflammation and structural changes. Cardiac ventricles, lungs, and sections of liver, spleen, kidney, skeletal muscle and gonad were stained with X-gal solution to evaluate non-target gene transfer. No blue coloration was observed on gross or microscopic analysis in any of these organs, indicating that the painting procedure was target specific.

H&E staining revealed epicardial atrial and ventricular adhesions and inflammation in all animals, regardless of treatment group (FIG. 1D). The inflammation was confined to the epicardium and was present in both painted and non-painted areas. Epicardial adhesions and inflammation were present at similar levels in control animals that only underwent open-chest manipulation of the heart. There was no association between adhesions or inflammation and the presence or type of virus, the presence of poloxamer, or the presence or concentration of trypsin, suggesting that the reaction was caused by the open-chest procedure rather than the gene transfer process. No evidence of intra-atrial fibrosis or loss of tissue integrity was noted in any animal.

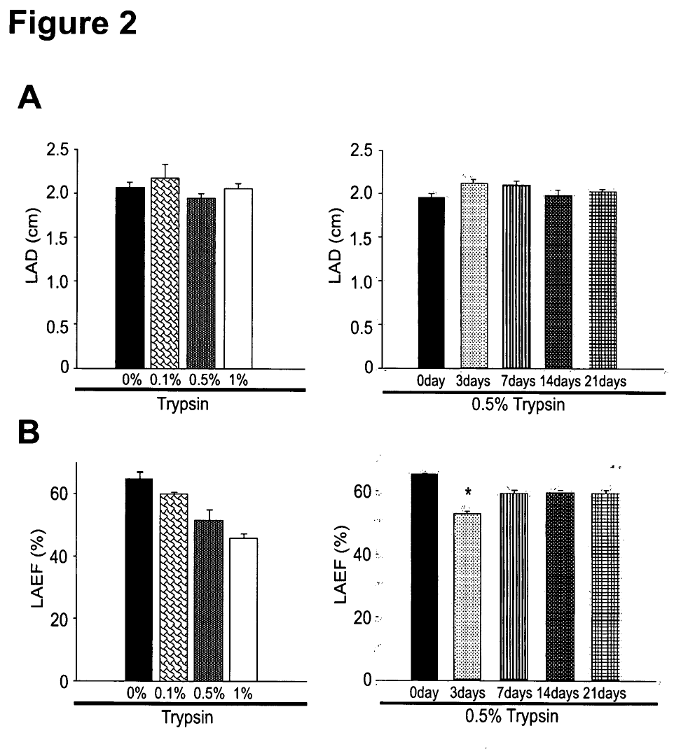

In the 3 day study, we examined the left atrial diameter (LAD) and left atrial ejection fraction (LAEF) by echocardiography one day prior to and 3 days after gene transfer. For the 21 day study, weekly echocardiograms were performed in addition to the pre-procedural and 3 day post-procedural studies. The results of these examinations showed no change in LAD for any of the animals (FIG. 2A: p=NS). Day 3 LAEF decreased in a dose-dependent manner. Even with 1% trypsin concentration though, the minimum LAEF was 45±1%, so moderate contractile function was maintained (FIG. 2B: p<0.05). For 0.5% trypsin exposure, serial measurements in the 21 day animals revealed that the decrease in LAEF was transient. There were no significant differences between pre-operative measurements and those taken on days 7, 14 or 21 (FIG. 2B: p=NS).

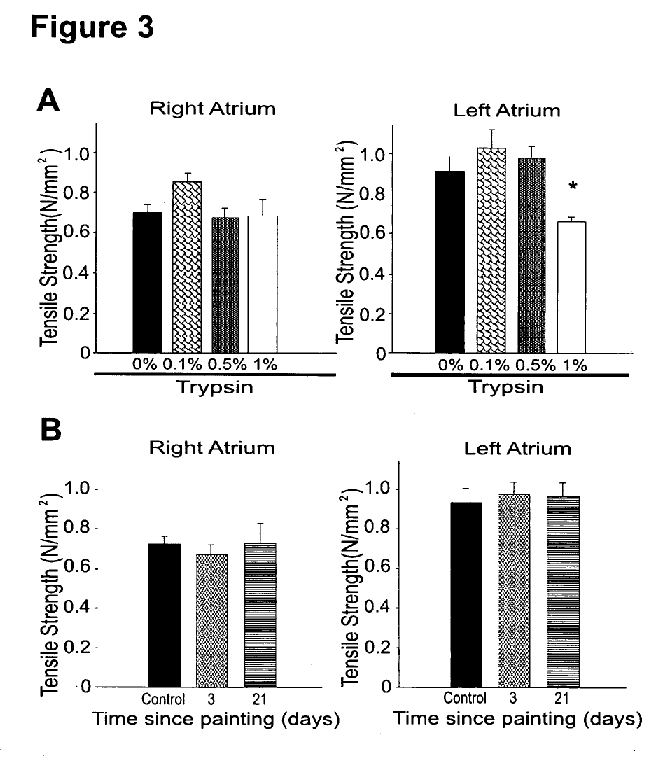

Structural integrity of the tissue was evaluated by the progressive addition of force to the tissue to define the breaking point. Strips of atria were cut to a uniform size and attached to the tensile strength apparatus. Force on the non-fixed end of the tissue was progressively increased at a fixed rate to define the breaking point of the strip of tissue. The tensile strength of right atrial myocardium was not changed significantly for any trypsin concentration. Left atrial tensile strength was unaffected for trypsin concentrations up to 0.5%, but there was a significant decrease in tensile strength for the 1% trypsin group (FIG. 3A: p<0.05). For the 0.5% trypsin exposure groups, there were no significant differences in tensile strength when compared to controls at 3 or 21 days post-gene transfer (FIG. 3B: p=NS).

### Example 4

### Modification of the Cardiac Phenotype by Gene Transfer with the Atrial Painting Method

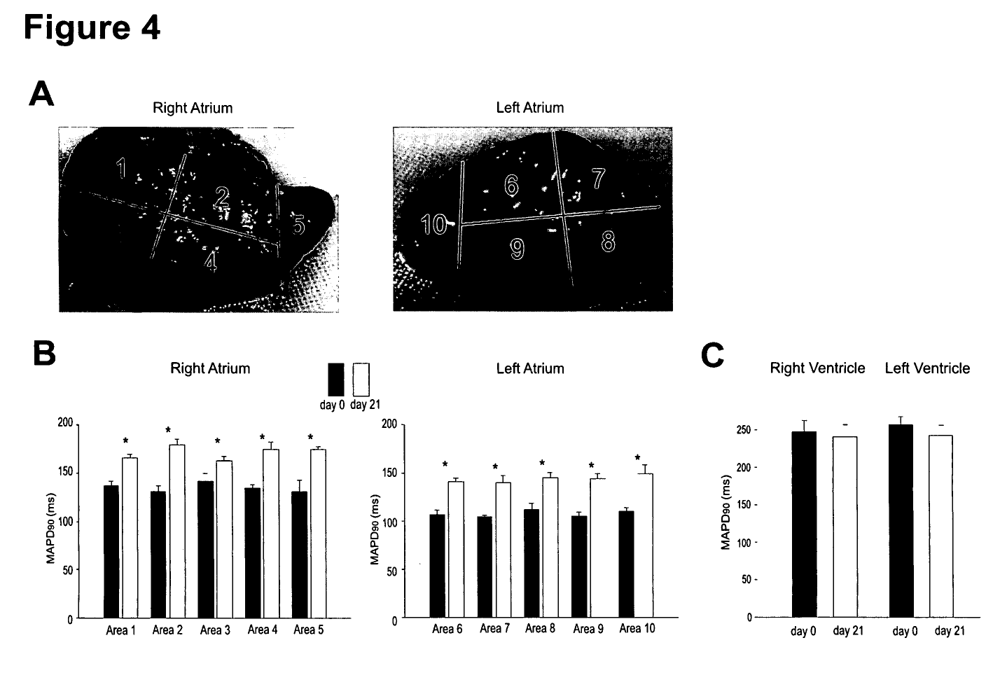

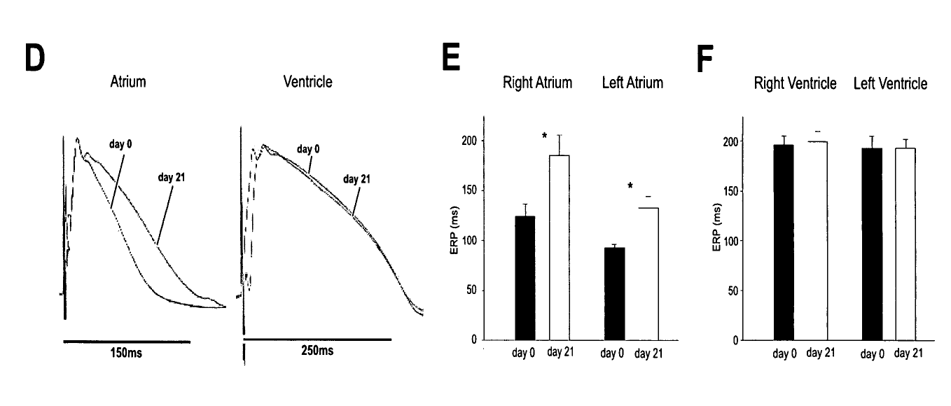

To evaluate functional effects achievable with this gene transfer method, we painted the atrial myocardium with AdHERG-G628S, encoding a dominant negative mutation for the rapid component of the delayed rectifier potassium current.15 Monophasic action potential duration (MAPD₉₀) and effective refractory period of atria and ventricles were measured before and 21 days after virus administration. Homogeneity of atrial effect was evaluated by measuring MAPD₉₀ in 10 predetermined regions spread in a grid-like pattern across both atria (FIG. 4A). Before gene transfer, MAPD₉₀ and atrial ERP were significantly longer in the RA compared to the LA, but no differences were found within each chamber. After 21 days, MAPD₉₀ in all 10 regions were uniformly prolonged 30±7% (FIG. 4B, p≦0.05). Differences in MAPD₉₀ of left versus right atria persisted after gene transfer. ERP in both atria were also increased by 48±18% (FIG. 4E, p≦0.01).

MAPD₉₀ and ERP were also measured in the basal region of the right and left ventricles to evaluate safety of the atrial gene transfer procedure. No changes were noted in MAPD₉₀ or ERP of the ventricles (FIGS. 4C and F, p=NS). In control animals receiving Adβgal, there were no changes in MAPD₉₀ or ERP for any cardiac chamber.

### REFERENCES

1. Tanigawa G, Jarcho J A, Kass S, Solomon S D, Vosberg H P, Seidman J G, Seidman C E. A molecular basis for familial hypertrophic cardiomyopathy: an alpha/beta cardiac myosin heavy chain hybrid gene. Cell. 1990;62:991-8.

2. Donahue J K, Heldman A W, Fraser H, McDonald A D, Miller J M, Rade J J, Eschenhagen T, Marban E. Focal modification of electrical conduction in the heart by viral gene transfer. Nat Med. 2000;6:1395-8.

3. Hajjar R J, Schmidt U, Matsui T, Guerrero J L, Lee K H, Gwathmey J K, Dec G W, Semigran M J, Rosenzweig A. Modulation of ventricular function through gene transfer in vivo. Proc Natl Acad Sci USA. 1998;95:5251-6.

4. Maurice J P, Hata J A, Shah A S, White D C, McDonald P H, Dolber P C, Wilson K H, Lefkowitz R J, Glower D D, Koch W J. Enhancement of cardiac function after adenoviral-mediated in vivo intracoronary beta2-adrenergic receptor gene delivery. J Clin Invest. 1999;104:21-9.

5. Losordo D W, Vale P R, Symes J F, Dunnington C H, Esakof D D, Maysky M, Ashare A B, Lathi K, Isner J M. Gene therapy for myocardial angiogenesis: initial clinical results with direct myocardial injection of phVEGF165 as sole therapy for myocardial ischemia. Circulation. 1998;98:2800-4.

6. Kay M A, Manno C S, Ragni M V, Larson P J, Couto L B, McClelland A, Glader B, Chew A J, Tai S J, Herzog R W, Arruda V, Johnson F, Scallan C, Skarsgard E, Flake A W, High K A. Evidence for gene transfer and expression of factor IX in haemophilia B patients treated with an AAV vector. Nat Genet. 2000;24:257-61.

7. Mann M J, Whittemore A D, Donaldson M C, Belkin M, Conte M S, Polak J F, Orav E J, Ehsan A, Dell'Acqua G, Dzau V J. Ex-vivo gene therapy of human vascular bypass grafts with E2F decoy: the PREVENT single-centre, randomised, controlled trial. Lancet. 1999;354:1493-8.

8. March K L, Madison J E, Trapnell B C. Pharmacokinetics of adenoviral vector-mediated gene delivery to vascular smooth muscle cells: modulation by poloxamer 407 and implications for cardiovascular gene therapy. Hum Gene Ther. 1995;6:41-53.

9. Feldman L J, Pastore C J, Aubailly N, Kearney M, Chen D, Perricaudet M, Steg P G, Isner J M. Improved efficiency of arterial gene transfer by use of poloxamer 407 as a vehicle for adenoviral vectors. Gene Ther. 1997;4:189-98.

10. Lonberg-Holm K, Philipson L. Early events of virus-cell interaction in an adenovirus system. J Virol. 1969;4:323-38.

11. Hardy S, Kitamura M, Harris-Stansil T, Dai Y, Phipps M L. Construction of adenovirus vectors through Cre-lox recombination. J Virol. 1997;71:1842-9.

12. Sanchez-Ramos J, Song S, Dailey M, Cardozo-Pelaez F, Hazzi C, Stedeford T, Willing A, Freeman T B, Saporta S, Zigova T, Sanberg P R, Snyder E Y. The X-gal caution in neural transplantation studies. Cell Transplant. 2000;9:657-67.

13. Franz M R. Method and theory of monophasic action potential recording. Prog Cardiovasc Dis. 1991;33:347-68.

14. Kovesdi I, Brough D E, Bruder J T, Wickham T J. Adenoviral vectors for gene transfer. Curr Opin Biotechnol. 1997;8:583-9.

15. Sanguinetti M C, Curran M E, Spector P S, Keating M T. Spectrum of HERG K+-channel dysfunction in an inherited cardiac arrhythmia. Proc Natl Acad Sci USA. 1996;93:2208-12.

16. Guzman R J, Lemarchand P, Crystal R G, Epstein S E, Finkel T. Efficient gene transfer into myocardium by direct injection of adenovirus vectors. Circ Res. 1993;73:1202-7.

17. Lamping K G, Rios C D, Chun J A, Ooboshi H, Davidson B L, Heistad D D. Intrapericardial administration of adenovirus for gene transfer. Am J Physiol. 1997;272:H310-7.

18. Kass-Eisler A, Falck-Pedersen E, Alvira M, Rivera J, Buttrick P M, Wittenberg B A, Cipriani L, Leinwand L A. Quantitative determination of adenovirus-mediated gene delivery to rat cardiac myocytes in vitro and in vivo. Proc Natl Acad Sci USA. 1993;90:11498-502.

19. Ikeda Y, Gu Y, Iwanaga Y, Hoshijima M, Oh S S, Giordano F J, Chen J, Nigro V, Peterson K L, Chien K R, Ross J, Jr. Restoration of deficient membrane proteins in the cardiomyopathic hamster by in vivo cardiac gene transfer. Circulation. 2002; 105:502-8.

20. March K L, Woody M, Mehdi K, Zipes D P, Brantly M, Trapnell B C. Efficient in vivo catheter-based pericardial gene transfer mediated by adenoviral vectors. Clin Cardiol. 1999;22:123-9.

21. Kabanov A V, Batrakova E V, Alakhov V Y. Pluronic block copolymers as novel polymer therapeutics for drug and gene delivery. J Control Release. 2002;82:189-212.

22. Lemieux P, Guerin N, Paradis G, Proulx R, Chistyakova L, Kabanov A, Alakhov V. A combination of poloxamers increases gene expression of plasmid DNA in skeletal muscle. Gene Ther. 2000;7:986-91.

23. Fromes Y, Salmon A, Wang X, Collin H, Rouche A, Hagege A, Schwartz K, Fiszman M Y. Gene delivery to the myocardium by intrapericardial injection. Gene Ther. 1999;6:683-8.

24. Chen H H, Mack L M, Kelly R, Ontell M, Kochanek S, Clemens P R. Persistence in muscle of an adenoviral vector that lacks all viral genes. Proc Natl Acad Sci USA. 1997;94:1645-50.

25. Mount J D, Herzog R W, Tillson D M, Goodman S A, Robinson N, McCleland M L, Bellinger D, Nichols T C, Arruda V R, Lothrop C D, Jr., High K A. Sustained phenotypic correction of hemophilia B dogs with a factor IX null mutation by liver-directed gene therapy. Blood. 2002;99:2670-6.

We claim:

1. A method of delivering a nucleic acid to an organ, comprising:
applying a composition to the external surface of the organ, wherein the composition comprises:
an adenovirus comprising the nucleic acid;
a poloxamer; and
trypsin.

2. The method of claim 1 wherein the poloxamer is F127.

3. The method of claim 1 wherein the poloxamer is L61.

4. The method of claim 1 wherein the poloxamer is present at a concentration of 5-30% in the composition.

5. The method of claim 1 wherein the poloxamer is present at a concentration of 15-25% in the composition.

6. The method of claim 1 wherein the poloxamer is present at a concentration of 20% in the composition.

7. The method of claim 1 wherein the trypsin is present at a concentration of 0.05% to 0.5% in the composition.

8. The method of claim 1 wherein the organ is a heart.

9. The method of claim 1 wherein the surface of the organ is an atrial epicardial surface.

10. The method of claim 1 wherein the nucleic acid encodes a protein and the protein is expressed in cells of the organ.

11. The method of claim 1 wherein the organ is a hollow organ.

12. The method of claim 1 wherein the organ is a gastrointestinal organ.

13. The method of claim 1 wherein the organ is a reproductive organ.

14. The method of claim 1 wherein the organ is selected from the group consisting of: stomach, gall bladder, small intestine, large intestine, rectum, uterus, and urinary bladder.

15. The method of claim 1 wherein the organ is selected from the group consisting of: eye, skin, diaphragm, and lung.

16. The method of claim 1 wherein the organ is in an animal's body.

17. The method of claim 1 wherein the organ is outside of an animal's body.

18. The method of claim 1 wherein the organ is a blood vessel.

19. A composition comprising:
a nucleic acid;
a poloxamer; and
a protease.

20. The composition of claim 19 wherein the poloxamer is F127.

21. The composition of claim 19 wherein the poloxamer is L61.

22. The composition of claim 19 wherein the poloxamer is present at a concentration of 5-30% in the composition.

23. The composition of claim 19 wherein the poloxamer is present at a concentration of 15-25% in the composition.

24. The composition of claim 19 wherein the poloxamer is present at a concentration of 20% in the composition.

25. The composition of claim 19 wherein the protease is trypsin.

26. The composition of claim 25 wherein the protease is present at a concentration of 0.05% to 0.5% in the composition.

27. The composition of claim 19 wherein the protease is chymotrypsin.

28. The composition of claim 19 wherein the nucleic acid is in a virus.

29. The composition of claim 28 wherein the virus is an adenovirus.

30. A kit for delivering a desired nucleic acid to an organ, comprising:
a poloxamer; and
a protease,
wherein the poloxamer and the protease are in a divided or undivided container.

31. The kit of claim 30 wherein the container is undivided.

32. The kit of claim 30 further comprising instructions for mixing the poloxamer and the protease with a nucleic acid.

33. The kit of claim 30 further comprising an instrument for painting the poloxamer and the protease on an external surface of an organ.

34. The kit of claim 33 wherein the instrument is a brush.

35. The kit of claim 33 wherein the instrument is a sponge.

36. The method of claim 9 wherein the nucleic acid comprises a dominant negative mutation for rapid component of delayed rectifier potassium current wherein the dominant negative mutation comprises a mutation in a HERG gene.

37. The method of claim 9 wherein the nucleic acid comprises a HERG-G628S allele.

38. The method of claim 19 wherein the nucleic acid comprises a dominant negative mutation for the rapid component of the delayed rectifier potassium current.

39. The method of claim 19 wherein the nucleic acid comprises a HERG-G628S allele.

40. The kit of claim 30 wherein the nucleic acid comprises a dominant negative mutation for the rapid component for the delayed rectifier potassium current.

41. The kit of claim 30 wherein the nucleic acid comprises a HERG-G628S allele.
